(19) United States
(12) Patent Application Publication
(10) Pub. No.: US 2012/0158051 A1
(43) Pub. Date: Jun. 21, 2012

(54) RE-TENSIONABLE KNOTLESS SUTURE SYSTEM

(76) Inventor: Seth A. Foerster, San Clemente, CA (US)

(21) Appl. No.: 13/330,026

(22) Filed: Dec. 19, 2011

Related U.S. Application Data
(60) Provisional application No. 61/424,479, filed on Dec. 17, 2010.

(51) Int. Cl. A61B 17/04

(52) U.S. Cl. 606/232

(57) ABSTRACT

Systems and methods for a knotless suture system, so that soft tissue may be secured to bone without the need of a suture knot. The system may include a bone anchor that engages bone tissue, and an eyelet through which a suture loop bundle may or may not pass. This bundle includes a snare and at least one loop of a tail of a length of suture that is secured to the soft tissue. The snare may have a variable diameter with at least two sections, a first one having a smaller dimension and a second section having a second, larger dimension and the suture loop bundle may pass through the eyelet when the snare includes the first, smaller section, but not the second section.

### CROSS-REFERENCE TO RELATED APPLICATIONS

This application claims the benefit under 35 U.S.C. §119(e) of U.S. Provisional Application No. 61/424,479 filed Dec. 17, 2010, the entirety of which is incorporated herein by reference in its entirety.

### BACKGROUND

This invention relates generally to methods and apparatus for attaching soft tissue to bone, and more particularly to apparatus and methods for securing connective tissue, such as ligaments or tendons, to bone. The invention has particular application to arthroscopic surgical techniques for reattaching the rotator cuff to the humeral head, in order to repair the rotator cuff. This invention relates more specifically to the creation of a sliding and locking loop of suture, and more particularly to a surgical technique of suturing and the formation of a suture loop that may be tightened, re-tensioned, and locked.

Suturing is a necessary aspect of virtually any surgical procedure. Numerous techniques of tying sutures have been developed by surgeons over the years to address various applications of sutures. In some cases, the development of a knot in a surgical procedure may require dexterity beyond the capability of the surgeon. This is certainly the case in surgeries such as arthroscopic, laparascopic, or thoroscopic surgery. A commonality in these procedures is that the spaces in which the surgeon works are limited and the tools used for suturing make tying knots difficult at best. Surgeons are accustomed to handling the suture, as knots in open procedures are typically tied and pushed down to the wound using the fingers. In endoscopic procedures, either the knots need to be tied externally to the body and inserted into the body and to the operative site using some kind of knot pushing device, or they need to be tied inside the body using long, clumsy instruments.

There have been other attempts to improve the methods of tissue repair. In orthopedic surgery, many different designs for bone anchors have been developed. These anchors allow soft tissues to be reattached to bone, and simplify the process by removing the need to create a transosseous tunnel. Less invasive arthroscopic techniques are beginning to be developed in an effort to address the shortcomings of open surgical repair. Working through small trocar portals that minimize disruption of the deltoid muscle, a few surgeons have been able to reattach the rotator cuff using various bone anchor and suture configurations. The rotator cuff is sutured intracorporeally and an anchor is driven into bone at a location appropriate for repair. Rather than thread the suture through transosseous tunnels which are difficult or impossible to create arthroscopically using current techniques, the repair is completed by tying the cuff down against bone using the anchor and suture.

However, as will now be described, there are cases where the knots themselves are a shortcoming in the effectiveness and easy of the procedure. In cases where joint re-constructions are undertaken by orthopedic surgeons, oftentimes the space available within joint is quite limited. This is especially true, for example, in a rotator cuff repair. The knots in the tendon can be difficult to tie and reposition as necessary, and may further be bulky and create a painful impingement of the tendon on the bone. So it may be seen that none of the currently extant approaches to the placement and securing of sutures in, for example, rotator cuff surgery have fulfilled all of the surgeon's requirements.

What is needed, therefore, is a new approach for providing knotless suture fixation to a bone anchor. Prior inventions have managed this with mechanisms that require activation through various linkages that have to be managed with a specialized handle. These inventions do not have the ability to re-tension the suture or re-position the suture within the suture lock once the lock has been secured. Other inventions require that the suture be pulled on the overcome rather significant friction in order to re-tension.

### SUMMARY

The present invention solves the problems outlined above by providing innovative connective techniques which permit a suture attachment to a bone anchor. In the present state of the art, the sutures which are passed through the tissues to be attached to bone typically are threaded through a small eyelet incorporated into the head of the anchor and then secured by tying knots in the sutures. Endoscopic knot tying is an arduous and technically demanding task, Therefore, the present invention discloses devices and methods for securing sutures to a bone anchor without the requirement of knot tying. The present invention provides systems and methods for securing soft tissue to bone, without the use of knots.

In one aspect of the invention a knotless suture system embodiment is disclosed comprising a bone anchor which is capable of engaging bone tissue, and this bone anchor includes an eyelet. A suture loop bundle may pass through this eyelet, the bundle including at least one loop of a suture tail and a snare. The suture tail is part of a length of suture that is secured to a piece of soft tissue such as a tendon, that is intended on being connected with the bone tissue. The snare is a variable diameter length of material, and has at least two portions, a first portion having a first dimension and a second portion having a second larger dimension. The eyelet cooperates with the suture loop bundle so that the suture loop bundle may pass though the eyelet when the bundle comprises the first portion of the snare, but may not pass through the eyelet when the suture loop bundle comprises the second snare portion.

Another embodiment of a knotless suture system according to the present disclosure comprises a bone locking mechanism with an adjustable eyelet and a suture loop bundle. The suture loop bundle may incorporate at least one loop from a suture tail, which is part of a length of a suture that is secured to a piece of soft tissue. The loop bundle also includes a snare. In this embodiment the eyelet is adjustable from a first position such that the suture loop bundle is free to pass through the eyelet, to a second position wherein the suture loop bundle is restricted from passing through the eyelet.

In another aspect of the disclosure a method for re-tensioning and locking a suture is disclosed, including providing a bone locking mechanism comprising an eyelet disposed at one end of the bone locking mechanism, followed by the step of threading a first portion of a snare through the eyelet. The snare has both a first and second diameter portion, and the first diameter portion is smaller than the second diameter portion. The method further comprises threading at least one free end from a length of suture through a snare loop to form a suture loop bundle, followed by pulling the suture loop bundle through the eyelet. The snare is then translated through the suture loop bundle such that the second diameter portion engages the suture loop bundle. The at least one free end of suture may then be pulled so as to bind the suture loop bundle and second diameter portion within the eyelet.

In a further aspect of the disclosure, a method for securing soft tissue with respect to a body cavity without knots is disclosed, comprising the steps of passing a length of suture through soft tissue so that a loop of suture material is embedded in the soft tissue resulting in two free ends. The distal end of the anchor body is then engaged with adjacent bone to fix the anchor body in place within the body cavity. A snare is then threaded through an eyelet in the anchor body to form a snare loop, the snare comprising a first and second diameter portion, wherein the first diameter portion is smaller than the second diameter portion. The method further comprises threading at least one free end through the snare loop to form a suture loop bundle, the snare loop comprising the first diameter portion, before pulling the suture loop bundle through the eyelet. The snare is then translated through the suture loop bundle such that the second diameter portion engages the suture loop bundle. The free end is then translated through the suture loop bundle to adjust the tension on the length of suture and this free end is then pulled to bind the suture loop bundle and second diameter portion within the eyelet.

The invention, together with additional features and advantages thereof, may best be understood by reference to the following description taken in conjunction with the accompanying illustrative drawing.

### BRIEF DESCRIPTION OF THE DRAWINGS

For a more detailed description of the preferred embodiment of the present invention, reference will be made to the accompanying drawings, wherein:

FIG. 1 shows a knotless suture system in accordance with at least some embodiments;

FIG. 2 shows a knotless suture system with suture tails assembled in accordance with at least some embodiments;

FIG. 3 shows the suture loop bundle passed through the eyelet of a knotless suture system in accordance with at least some embodiments;

FIG. 4 shows the second portion of the snare of a knotless suture system translated so as to engage the suture loop bundle in accordance with at least some embodiments;

FIG. 5 shows the suture loop bundle engaged with eyelet of a knotless suture system to lock or bind the sutures in accordance with at least some embodiments;

FIG. 6 shows a configuration of a knotless suture system prior to cutting the sutures and snares in accordance with at least some embodiments;

FIGS. 7a-7c and 8a-8b illustrate the knotless suture system in accordance with at least some embodiments;

FIGS. 9a-9c illustrate the knotless suture system in accordance with at least some embodiments;

FIGS. 10a-10b illustrate a bone locking mechanism eyelet in accordance with at least some embodiments; and

FIGS. 11a-11c illustrates a bone locking mechanism eyelet in accordance with at least some embodiments.

### DETAILED DESCRIPTION OF THE PREFERRED EMBODIMENTS

In the description that follows, like parts are marked throughout the specification and drawings with the same reference numerals, respectively. The drawing figures are not necessarily to scale. Certain features of the invention may be shown exaggerated in scale or in somewhat schematic form, and some details of conventional elements may not be shown in the interest of clarity and conciseness. The present invention is susceptible to embodiments of different forms. There are shown in the drawings, and herein will be described in detail, certain embodiments of the present invention with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results.

The present invention provides improved methods and devices for knotless suturing of tissue. Although the variation discussed herein discusses use of a suture, the term “suture” may include any piece of material that is used to close a wound or connect tissue (e.g., catgut, thread, wire, etc.) so long as the material can be used with the other portions of the system as described herein. Accordingly, sutures as described herein may include polymeric, metallic, or other types of sutures.

For illustrative purposes, the examples discussed herein describe the use of an anchoring system to suture soft tissue to a bone structure. In one variation of the system, the medical practitioner affixes a length of suture through soft tissue to approximate and fix the soft tissue with respect to the body cavity (e.g., a bored hole in the bone structure). It should be understood, however, that the suture anchor apparatus may be utilized to secure a length of suture to body cavities other than in a bone structure, and may even be used to anchor the suture outside of a body cavity, merely to a predetermined location within the body.

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

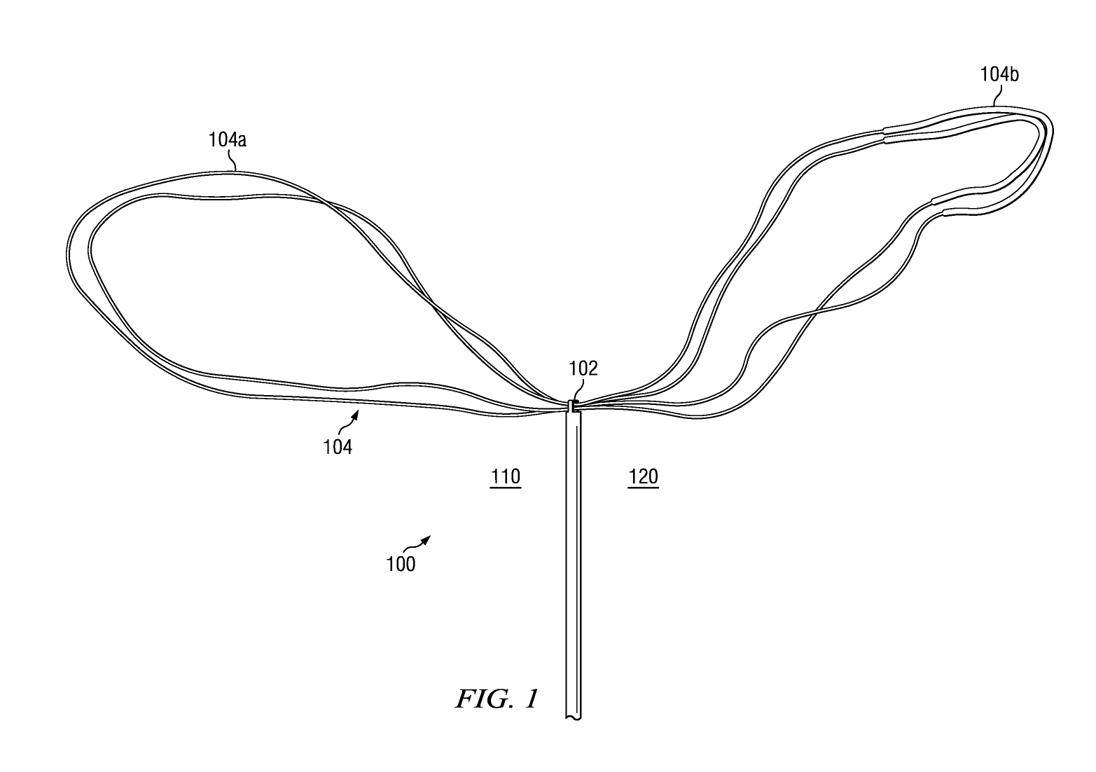

FIG. 1 illustrates a portion of a knotless suture system 100 in accordance with at least some embodiments. Knotless suture system 100 comprises an eyelet 102 and a snare 104. In certain embodiments, the eyelet 102 is part of a bone anchoring system that may include a bone locking or bone anchoring feature such as a threaded screw or barbed structure (not shown here). The threads or barbs may engage the bone or a bone tunnel and this bone anchoring feature may be disposed adjacent and often distal to the eyelet 102. Snare 104 may be a length of material, such as a suture or other elongate flexible structure such as a ribbon or wire. It may be a continuous loop as shown in FIG. 1, but may also have two free ends (not shown here). Snare 104 has at least one smaller diameter portion 104a and at least one portion that is significantly larger in diameter 104b. In alternative embodiments snare 104 may not have a circular cross section and in these embodiments, snare 104 may have at least one portion 104a, that has a smaller coincident dimension compared with a second portion dimension.

There may be several first and second portions along the length of snare and there may be some indicators such as color changes or lines to indicate the different portions (not shown here). Snare 104 may be one continuous loop, or, as shown here multiple loops of material. Transitions between first and second portions may be abrupt or gradual. Abrupt transitions may be more apparent to the user, but may increase the likelihood of snagging during the translation of the snare (described later). In alternate embodiments snare 104 may also have portions with varying flexibility and portions with lower flexibility. These differing flexibilities may be as a result of changes in materials or as a result of varying cross sections of the snare 104.

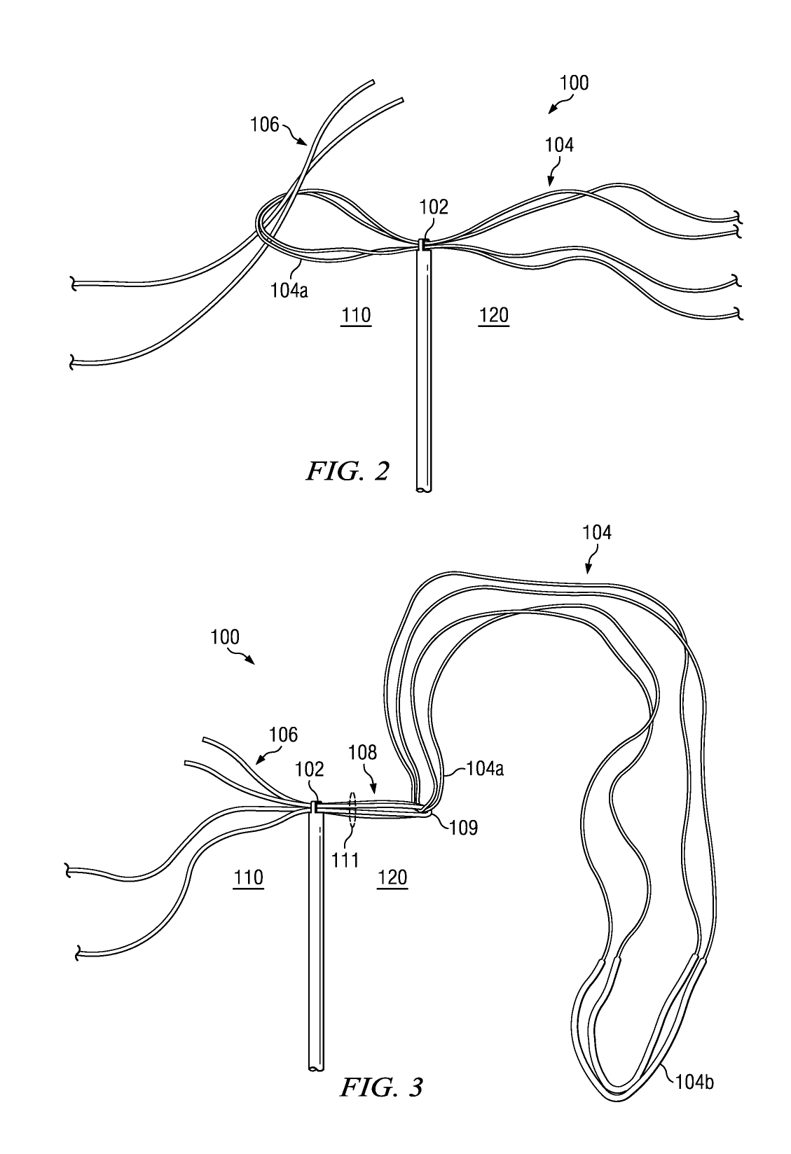

Generally the system relies on the eyelet 102 and snare 104 being sized so as to allow the passage of the smaller snare portion 104a through the eyelet 102, together with an object that has been snared. The eyelet 102 is further operable so that a snared object together with the second portion 104b may not have passage through the eyelet 102. As shown in FIG. 2, the ensnared object may be two tails 106, connected to a piece of soft tissue (not shown here).

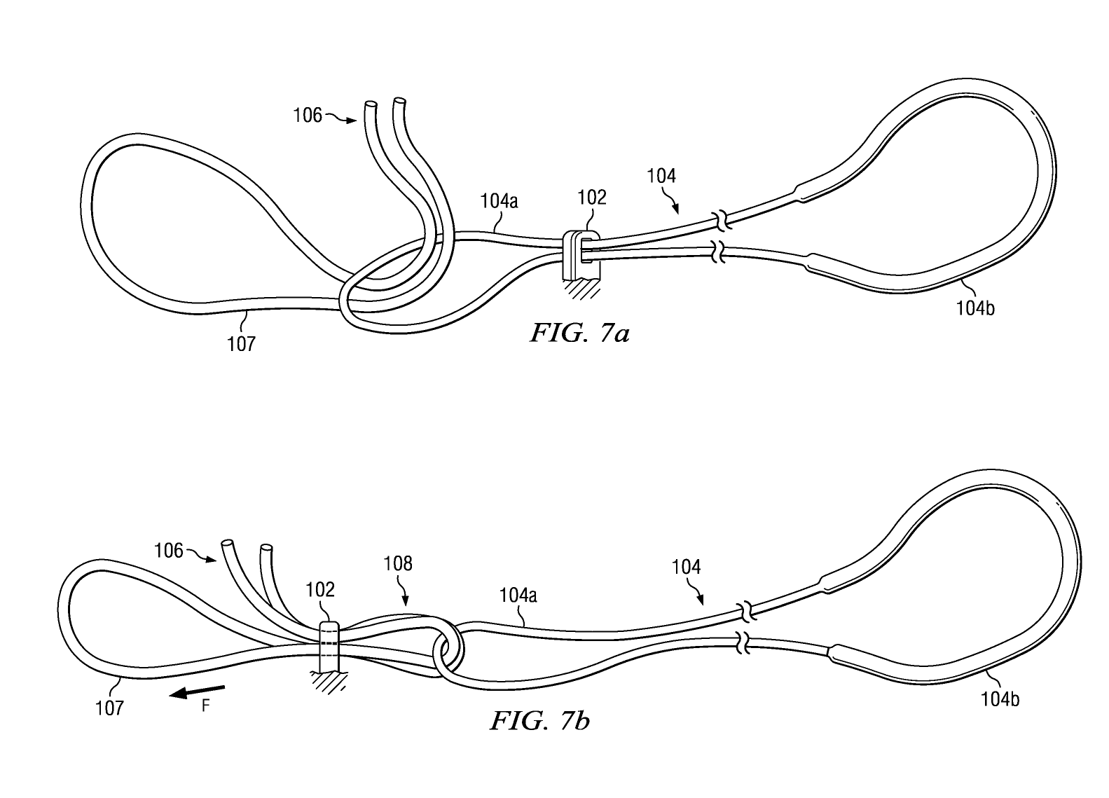

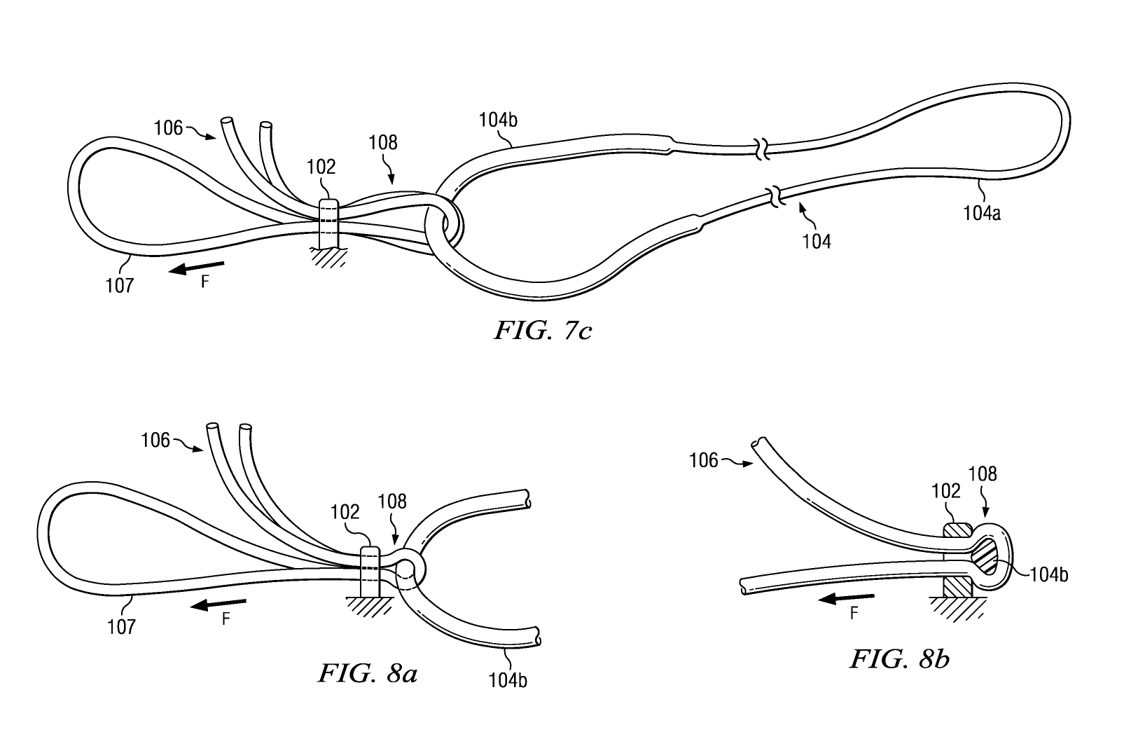

In one embodiment, a suture tissue loop 107 is formed through a tissue or graft (see FIG. 7) resulting in two suture tails 106 that are to be snared. Tails 106 may then be threaded through the snare 104 on the tissue side 110 of the eyelet to form a suture loop bundle 108, as illustrated in FIGS. 2, 3, and 7b. The snare 104 pulls the suture bundle 108 into the eyelet 102 so that the bundle 108 is moved to the snare side 120 of the eyelet and the suture tails have formed two suture tail loops 111. Only a short segment of suture tails 106 may be required to be placed through eyelet 102, thereby saving the physician procedure time. Further, the suture tails 106 may be threaded without an insertion device, saving on instrumentation and potentially procedure time. At this point in time, there are four portions (two loops 111) of the tissue suture in the eyelet 102 and the snare 104 is holding the apex 109 of this bundle 108 in close proximity to the eyelet 102, as illustrated in FIG. 3. As described earlier, the eyelet 102 is of such a size that it barely lets the suture bundle 108 through with its snare 104. The snare 104 is now rotated or translated through the apex 109 of the suture bundle 108 so that the second snare portion 104b is present in the apex. Because of this larger snare profile, the suture bundle 108 cannot be pulled back through the eyelet 102 to the tissue side 110.

The knotless mechanism is now assembled. The larger snare material portion 104b rests against the eyelet 102 and acts like the tension bar in a buckle. Referring now to FIGS. 5, 7b-7c, and 8a-8b, a force F applied to suture tissue loop 107 in addition to the action of pulling the suture tails 106 pulls the suture loop bundle 108 into the eyelet 102 and binds up the suture apex 109 in the eyelet 102 so that it binds and locks the suture.

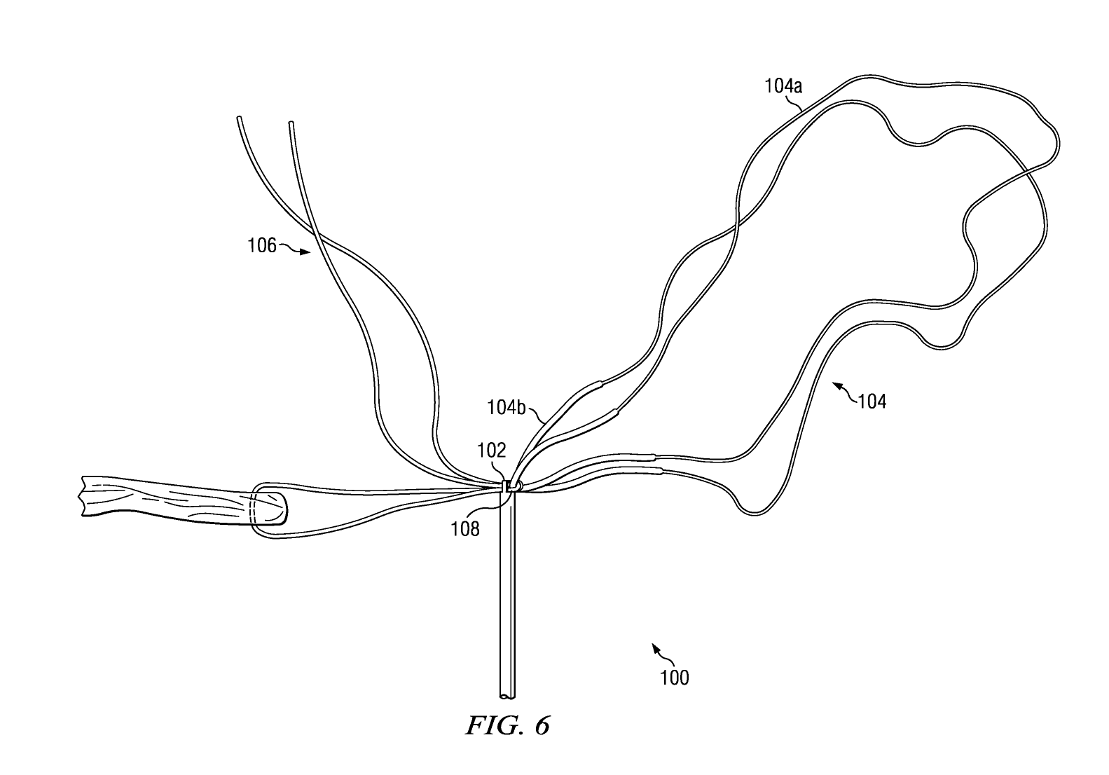

The tension on the suture loop 107 may now also be altered. By applying tension on the suture tails 106 in conjunction with applying sufficient tension on the snare 104, the locking configuration of the apex 109 within the eyelet may be released and the suture loop 107 may slide through the apex 109 so as to further draw the tissue towards the eyelet 102. Alternatively, tension may be applied to the suture loop 107 in conjunction with applying sufficient tension on the snare 104 and the tissue may be allowed to move away from the eyelet 102. By so pulling on snare 104 with sufficient force, the locking configuration of the suture within the eyelet 102 is released such that the suture apex 109 is pulled away from eyelet 102. This allows slack for the suture tails 106 or suture loop 107 to be pulled. It has been found that this back and forth motion to increase tension on the loop 107 sets the suture tails 106 in the top of the eyelet 102 and the suture loop 107 in the bottom of the eyelet 102 as is necessary for a proper lock. Once the tissue is in its desired position, the suture tails 106 and the snare 104 as shown in the configuration illustrated in FIG. 6 can be severed with any sharp instrument.

The eyelet 102 may be made of any material including plastic, metal, ceramic, bio-absorbable, and bio-resorbable. The snare 104 may be constructed from plastic or metal material. Plastic would be preferred as it is easier to sever at the end of the procedure. The top (proximal end of) eyelet 102 is preferred to be about the same width as two suture diameters or dimensions. The base of the eyelet 102 may be larger. The locking diameter or dimension of the snare material 104b may be larger than the diameter of the tissue suture loop 107. Additionally, the larger section of snare material 104b may be made of any cross-section, such as a braided suture. Additionally, triangular, rectangular, or eccentric cross-section shapes for the larger section of snare material 104b are contemplated.

FIGS. 7a-7c and 8a-8b provide additional illustration of the knotless suture system in accordance with the embodiment described above. As illustrated and described in FIGS. 7a-7c and 8a-8b, a method in accordance with these embodiments may include: providing a bone locking mechanism comprising an eyelet 102 disposed at one end of the bone locking mechanism; threading a snare 104 through the eyelet 102, the snare 104 comprising a first and second diameter material (104a, 104b), wherein the first diameter material 104a is smaller than the second diameter material 104b; threading at least two free ends 106 from a length of suture through a snare loop to form a suture loop bundle 108, the snare loop comprising the first diameter material; pulling the suture loop bundle 108 through the eyelet 108; translating the snare 104 through the suture loop bundle 108 such that the second diameter material 104b engages the suture loop bundle 108; pulling the at least two free ends 106 of suture to bind the suture loop bundle 108 and second diameter material 104b within the eyelet 102; and pulling the snare 104 to release the suture loop bundle 108 from the eyelet 102 to allowing re-tensioning.

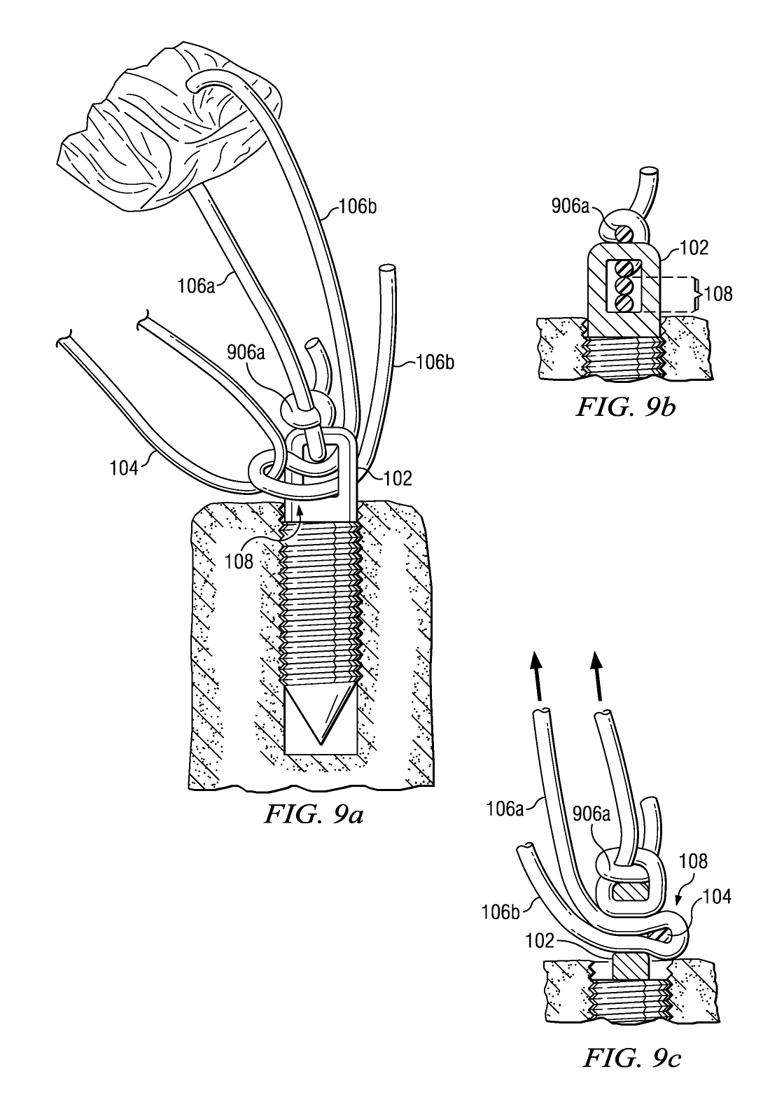

Referring now to FIGS. 9a-9c, an alternative embodiment of a knotless suture system is shown. In this embodiment, one of suture the tails 106a is looped through eyelet 102 and secured thereto with a knot 906a. The second of the suture tails 106b is routed through eyelet 102 to form a loop 105 similar to the embodiment described formerly, and functions as the tensioning suture tail. A portion of the second suture tail 106b (loop 105) is used to form the suture loop bundle 108, together with snare 104, which engages with and is routed through suture loop bundle 108, similar in fashion to the embodiment describes previously. In this configuration, the profile of eyelet 102 in which the suture lock occurs may be significantly smaller, thereby also reducing the cinching forces required. A benefit of this configuration is that the likelihood of the suture 106 pulling out of the eyelet 102 during tensioning may be reduced.

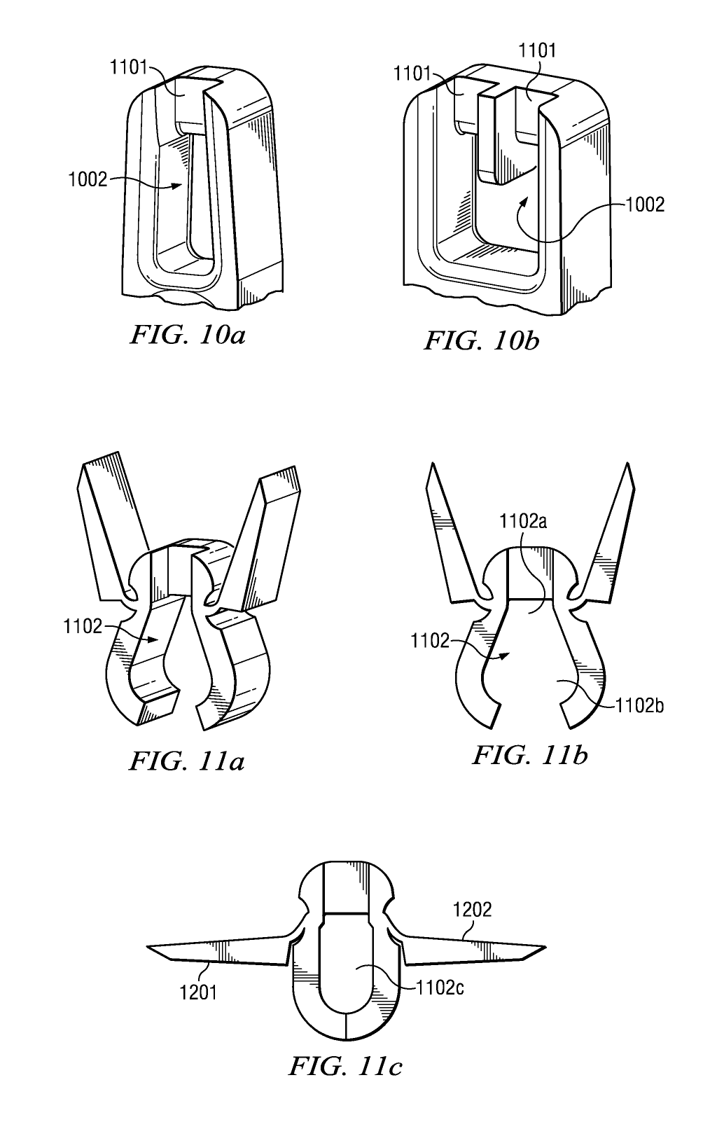

Referring now to FIGS. 10a-10b, an embodiment of a knotless suture eyelet 1002 is illustrated. In this embodiment, eyelet 1002 may be formed at the top of a bone locking feature. Eyelet 1002 is characterized by at least one groove 1101 disposed at the top of the eyelet 1002. When utilized in a knotless suture system configuration such as is illustrated in FIGS. 9a-9c, groove 1101 assists in achieving a stable functional configuration of the locking suture bundle, similar to the bundles described earlier (not shown here). In particular, it has been discovered that the locking suture bundle configuration works best when the tensioning tail 106 or 106b is kept above the tissue tail 106a or tissue loop 107. For example, when the knotless suture system is loaded in its functional suture locking configuration, the tensioning tail 106b is slack while the tissue tail 106a is loaded under a tensioned force. However, in some instances the tensioning tail 106b tends to slip above the tissue tail 106a, which can inadvertently release the tensioning force on tissue tail 106a. The groove 1101 shown in eyelet 1002 in FIG. 10a, for example, retains the tail 106b in position under the tensioned tissue tail 106a and thereby enables the binding suture locking bundle contemplated herein.

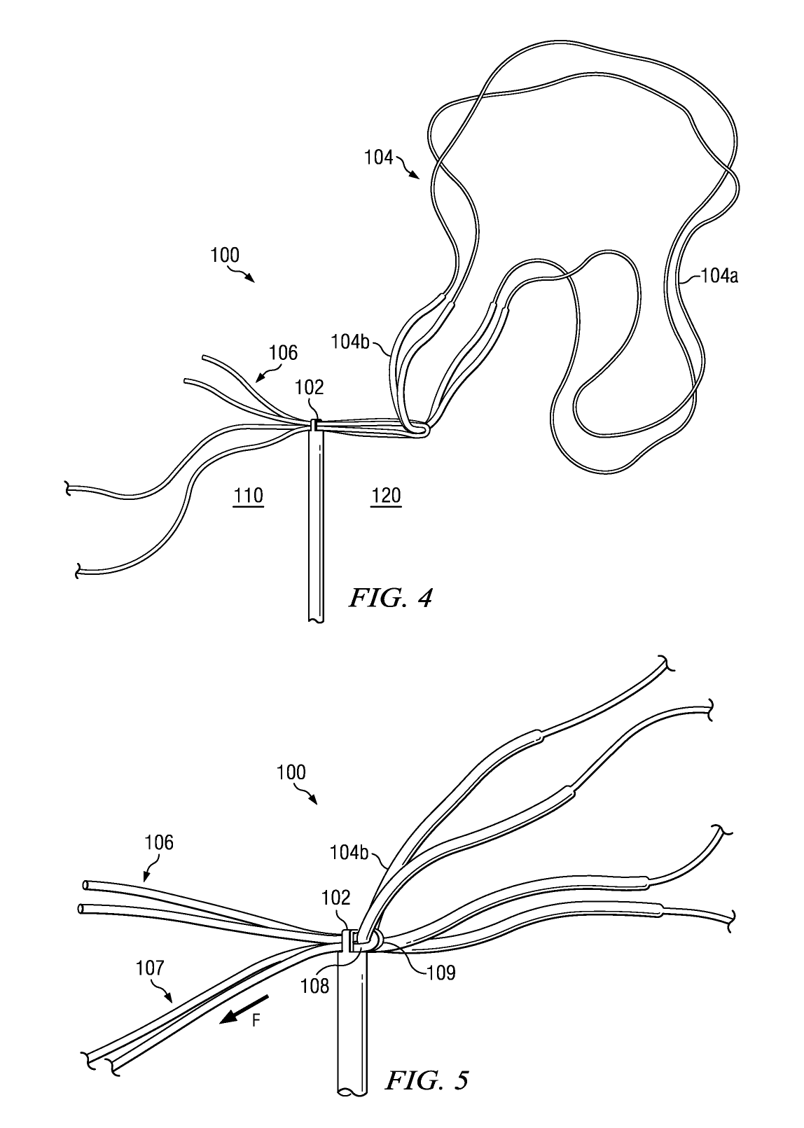

Referring now to FIG. 11a-11c, an embodiment of a bone locking mechanism eyelet is illustrated, which may be used in a fashion similar to that described in earlier embodiments. In this embodiment, eyelet 1102 is collapsible from an open configuration with a large opening 1102b at the bottom and a small opening 1102a at the top, to a second, closed or collapsed configuration which may have a more uniform opening 1102c. The eyelet 1102 is initially placed in the open configuration where a suture bundle is threaded in the large opening, allowing for the snare material to be big (i.e., snare portion 104b in FIGS. 1-8b) rather than the required smaller dimension snare (i.e., snare portion 104a in FIGS. 1-8b) in order to thread the eyelet 102 as described above. The suture bundle is pulled up into the small opening 1102a at the top to trap the apex of the suture bundle in that side of the eyelet 1102. Small opening 1102a may be sized so as to be smaller than the snare or suture dimensions so that the bundle is somewhat trapped into position. The eyelet 1102 may be collapsed to the uniform configuration 1102c by deploying bone lock arms 1201, 1202. In this deployed configuration, the apex is completely trapped, no matter where it is located within the eyelet. Eyelet may be reversibly collapsed so as to be able to re-tension the length of tissue suture loop. Eyelet 1102 may have at least one groove 1101, disposed adjacent a portion of eyelet 1102 to nest the suture tail as described above.

While preferred embodiments of this invention have been shown and described, modifications thereof can be made by one skilled in the art without departing from the scope or teaching herein. The embodiments described herein are exemplary only and are not limiting. Because many varying and different embodiments may be made within the scope of the present teachings, including equivalent structures or materials hereafter thought of, and because many modifications may be made in the embodiments herein detailed in accordance with the descriptive requirements of the law, it is to be understood that the details herein are to be interpreted as illustrative and not in a limiting sense.

What is claimed is:

1. A knotless suture system comprising:
a bone anchor operable to engage bone tissue, said bone anchor having an eyelet;
a suture loop bundle adjacent the eyelet, comprising at least one loop of a suture tail and a snare, the snare comprising at least one first portion having a first dimension and at least one second portion having a second dimension, wherein the first portion is smaller than the second portion.

2. The knotless suture system of claim 1, wherein the suture loop bundle may pass though the eyelet when the bundle comprises the first snare portion.

3. The knotless suture system of claim 1, wherein the suture loop bundle may not pass through the eyelet when the suture loop bindle comprises the second snare portion.

4. The knotless suture system of claim 1, wherein the bone anchor comprises threads or barbs to engage the bone tissue.

5. The knotless suture system of claim 1, wherein the suture tail is connected to soft tissue.

6. The knotless suture system of claim 1, wherein a second suture tail, connected with the first suture tail, is fixed to a portion of the bone anchor.

7. The knotless suture system of claim 1, wherein the suture loop bundle comprises at least two suture tail loops.

8. The knotless suture system of claim 1, wherein the eyelet further comprises at least one groove operable to nest the at least a portion of the suture tail within the groove during operation.

9. A knotless suture system comprising:
a bone locking mechanism comprising an adjustable eyelet;
a suture loop bundle comprising at least one loop from a suture tail and a snare, wherein the eyelet is adjustable from a first position such that the suture loop bundle is free to pass through the eyelet, to a second position wherein the suture loop bundle is restricted from passing through the eyelet.

10. The knotless suture system of claim 9 wherein the bone locking mechanism comprises at least one bone locking arm, operable to collapse the eyelet to the second position.

11. The knotless suture system of claim 9, wherein the eyelet further comprises a first eyelet portion that is smaller than a second eyelet portion.

12. The knotless suture system of claim 9, wherein the eyelet is a uniform shape once in the second position.

13. The knotless suture system of claim 9, wherein the at least one suture tail is secured to soft tissue.

14. The knotless suture system of claim 9, wherein a second suture tail, connected with the first suture tail, is fixed to a portion of the bone locking mechanism.

15. The knotless suture system of claim 9, wherein the eyelet further comprises at least one groove operable to nest a portion of the suture tail within the groove during operation.

16. A method for re-tensioning and locking a suture comprising:
providing a bone locking mechanism comprising an eyelet disposed at one end of the bone locking mechanism;
threading a snare through the eyelet, the snare comprising a first and second diameter portion, wherein the first diameter portion is smaller than the second diameter portion;
threading at least one free end from a length of suture through a snare loop to form a suture loop bundle, the snare loop comprising the first diameter portion;
pulling the suture loop bundle through the eyelet;
translating the snare through the suture loop bundle such that the second diameter portion engages the suture loop bundle; and
pulling the at least one free end of suture to bind the suture loop bundle and second diameter portion within the eyelet.

17. The method of 16 further comprising the step of pulling the snare to release the suture loop bundle from the eyelet to release the at least one free end.

18. The method of 17 further comprising the step of sliding the at least one free end of suture through the suture loop bundle to adjust the tension on the length of suture.

19. The method of 18 further comprising the step of pulling the at least one free end of suture to bind the suture loop bundle within the eyelet.

20. A method of securing soft tissue with respect to a body cavity without knots, comprising:
passing a length of suture through soft tissue so that a loop of suture material is embedded in the soft tissue resulting in two free ends;
engaging a distal end of the anchor body with adjacent bone to fix the anchor body in place within the body cavity;
threading a snare first portion through an eyelet in the body anchor, the snare comprising both a first and second portion, and wherein the first portion is smaller in diameter than the second portion;
threading at least one free end through a snare loop to form a suture loop bundle;
pulling the suture loop bundle through the eyelet;
translating the snare through the suture loop bundle such that the second diameter portion engages the suture loop bundle;
translating the at least one free end through the suture loop bundle to adjust the tension on the length of suture; and
withdrawing the at least one free end of suture together with the length of suture to bind the suture loop bundle and second diameter portion within the eyelet.

21. A method for locking a suture comprising:
providing a bone locking mechanism comprising an adjustable eyelet disposed at one end of the bone locking mechanism;
threading a snare loop through the eyelet, the eyelet comprising a first portion proximal to a second portion, wherein the first portion has a smaller opening than the second portion;
threading at least one free end from a length of suture through a snare loop to form a suture loop bundle;
pulling the suture loop bundle through the eyelet;
placing an apex of the suture loop bundle within the first portion of the eyelet; and
collapsing the eyelet to cinch the suture loop bundle within the eyelet.
